(19) United States
(12) Patent Application Publication
(10) Pub. No.: US 2026/0341525 A1
(43) Pub. Date: Oct. 29, 2026

(54) AI-derived biomarker measuring relative size of pulmonary arterial tree compared to the lungs

(71) Applicant: University of Iowa Research Foundation, Iowa City, IA (US)

(72) Inventors: Seyed Soheil Hosseini, Iowa City, IA (US); Eric A. Hoffman, Iowa City, IA (US)

(73) Assignee: University of Iowa Research Foundation, Iowa City, IA (US)

(21) Appl. No.: 19/657,712

(22) Filed: Apr. 24, 2026

Related U.S. Application Data
(60) Provisional application No. 63/793,747, filed on Apr. 24, 2025.

(51) Int. Cl. G16H 50/30; G06N 3/0442; G16H 30/40; G16H 50/70

(52) U.S. Cl. CPC G16H 50/30; G06N 3/0442; G16H 30/40; G16H 50/70

(57) ABSTRACT

A method for assessing pulmonary vascular characteristics includes obtaining medical imaging data of a subject's lungs, segmenting pulmonary arterial structures from the medical imaging data, calculating a pulmonary-arterial-to-lung ratio (ArtLR) by determining a relationship between arterial dimensions at predetermined anatomical locations and lung volume, and using the calculated ArtLR as a biomarker for assessing pulmonary structure and function.

### RELATED APPLICATIONS

This application claims priority to U.S. Provisional Patent Application No. 63/793,747, filed Apr. 24, 2025, hereby incorporated by reference in its entirety.

### STATEMENT REGARDING FEDERALLY FUNDED RESEARCH

This invention was made with government support under HL126838, HL137880, and 75N92024D00012 awarded by the National Institutes of Health. The government has certain rights in the invention.

### FIELD OF THE DISCLOSURE

The present disclosure relates to AI-derived biomarkers. More particularly, but not exclusively, the present invention relates to AI-derived biomarker measuring relative size of pulmonary arterial tree compared to the lungs.

### BACKGROUND

Chronic Obstructive Pulmonary Disease (COPD) and related respiratory disorders represent significant public health concerns worldwide, particularly in populations with smoking history. Current diagnostic and prognostic methods for these conditions remain limited in their ability to fully characterize the relationship between pulmonary structure and function, particularly regarding vascular contributions to disease pathophysiology.

Prior research has established that an airway dysanapsis (size-mismatch) index (Airway-to-Lung Ratio: AirLR) can be assessed as a measure of mismatch between airway size and lung volume. This index is calculated from the average cross-sectional area at 24 standard locations on the airway tree divided by the cube root of lung volume. Studies have demonstrated that this index strongly predicts risk of COPD regardless of smoking status and serves as a significant risk factor for other pulmonary pathologies (Smith BM, et al. JAMA. 2020; 323(22): 2268-2280).

Further research has shown that total pulmonary vascular volume-to-lung volume is smaller in individuals with airway dysanapsis (Gerard SE, et al. Ann Am Thorac Soc. 2024). However, due to the close developmental relationship between bronchial and arterial trees, there remains an unmet need to evaluate a measure of pulmonary arterial tree dysanapsis (Arterial-to-Lung Ratio: ArtLR) relative to AirLR.

Although the significance may not be apparent without the benefit of this disclosure, evidence suggests that in areas of inflammation, emphysema risk is associated with hypoxic-induced peripheral arterial constriction, where constriction can be indexed by segmental-level arterial enlargement. This observation further motivates the need for precise segmentation, characterization, and measurement of the pulmonary arterial tree.

Current methods for segmentation and characterization of pulmonary arterial trees from CT scans face several technical challenges. These include difficulty in distinguishing between pulmonary arteries and veins in CT images, particularly in contrast-enhanced dual-energy CT (DECT) scans. These further include lack of standardized methods for identifying consistent locations on arterial trees across different subjects for comparative analysis. If airways are to be used as a guide for establishing standard locations on the arterial trees, this will further include challenges in matching corresponding arterial and airway segments due to anatomical variations and complex branching patterns. These further include absence of automated methods for calculating an arterial dysanapsis index analogous to the established airway dysanapsis index. Challenges further include limited understanding of the relationship between arterial tree characteristics and various pulmonary function or structural biomarkers.

Current approaches to addressing these challenges typically involve manual or semi-automated segmentation of vascular structures, which is extremely time-consuming, prone to inter-observer variability, and difficult to scale for larger population studies.

Furthermore, existing methods often fail to properly account for the complex three-dimensional structure and branching patterns of pulmonary arterial trees. The arterial tree structure presents particular challenges for segmentation algorithms due to varying diameters, orientations, and branching topologies across different subjects and lung regions.

There is thus a critical need for automated methods that can accurately segment pulmonary arterial trees from CT scans, identify standardized locations for measurement, characterize tree morphology, and calculate indices that can be correlated with pulmonary functional and structural biomarkers to enable applications such as, but not limited to, improved understanding of COPD pathophysiology or other conditions as well as potentially guide therapeutic interventions.

Therefore, what is needed is an automated system for accurate segmentation and standardized characterization of pulmonary arterial trees.

### SUMMARY

The methods and systems described herein address a technical problem that is specifically rooted in medical imaging technology: the inability of existing computerized pulmonary imaging pipelines to reliably segment, label, and quantitatively compare pulmonary arterial tree structures across subjects in a reproducible, subject-size-invariant manner. Prior computerized approaches fail in concrete technical ways. For example, they cannot reliably distinguish arteries from veins in close spatial proximity (particularly in contrast-enhanced dual-energy CT), cannot establish consistent standard measurement locations across subjects with differing anatomy and lung size, cannot robustly match corresponding airway and arterial segments where branching topology diverges between subjects, and cannot compensate for the distortion of vascular geometry that occurs at different lung inflation volumes (TLC versus FRC). These are technical limitations of image-processing pipelines operating on three-dimensional CT volumes.

The disclosed embodiments provide specific technical solutions to each of these problems that improve the functioning of computerized pulmonary imaging systems themselves. Deep-learning segmentation networks (including U-Net and latent-diffusion architectures) are trained on curated artery/vein-labeled datasets to separate spatially adjacent vascular structures that conventional thresholding and region-growing methods cannot resolve. Diameter-defined Strahler ordering iteratively refined against measured branch diameters yields non-overlapping, subject-size-invariant branch orders that enable consistent selection of standard measurement locations across a population. An LSTM-based matching network operating on a multi-dimensional geometric feature vector (proximity, relative orientation, generational position, branch-length ratio, branch-diameter ratio, and airway segment identifier) resolves the ambiguous one-to-many and zero-match correspondences between airway and arterial segments that defeat hand-crafted rule-based matchers. Acquiring airways at TLC and arteries at FRC, then warping TLC-space airway labels into FRC space via image registration, compensates for inflation-dependent vascular distortion in a way that pure image-domain processing cannot. The resulting arterial-to-lung ratio is a concrete, measurable output of the imaging pipeline, not an abstract correlation, and may be applied in a practical application: phenotyping patients for clinical-trial enrollment, stratifying COPD risk, and guiding therapeutic selection. These improvements are necessarily rooted in computerized medical-imaging technology and produce a specific, quantifiable improvement in the reliability, reproducibility, and cross-subject comparability of pulmonary vascular assessment.

Therefore, it is a primary object, feature, or advantage of the present invention to improve over the state of the art.

It is a further object, feature, or advantage to provide a measure of arterial tree size relative to lung size.

It is a further object, feature, or advantage of the present disclosure to provide automated methods for segmenting anatomical structures within the chest.

It is a still further object, feature, or advantage of the present disclosure to generate structured representations of anatomical trees to support consistent analysis across individuals.

Another object, feature, or advantage is to define and compute indices relating anatomical structures to organ size, enabling quantitative assessment of developmental or pathological mismatches.

Yet another object, feature, or advantage is to support correlation of anatomical measurements with functional, structural, or clinical data to enhance diagnostic and prognostic capabilities.

It is a further object, feature, or advantage of the present disclosure to improve the reproducibility and scalability of anatomical assessments across large populations.

It is a still further object, feature, or advantage of the present disclosure to facilitate the identification of standard measurement locations within complex branching structures.

Another object, feature, or advantage is to provide a framework for comparing anatomical variability and asymmetry within or between individuals.

Yet another object, feature, or advantage is to enable integration of anatomical indices into clinical research and decision support systems.

It is a further object, feature, or advantage of the present disclosure to support training, validation, and refinement of computational models using curated anatomical data.

It is a further object, feature, or advantage of the present disclosure to differentiate between pulmonary arterial and venous structures using artificial intelligence techniques.

Another object, feature, or advantage is to provide methods for matching corresponding structures between airway and arterial trees using machine learning and neural network approaches.

Yet another object, feature, or advantage is to offer multiple standardization approaches that can be selected based on specific clinical applications or imaging conditions.

It is a further object, feature, or advantage of the present disclosure to enable phenotyping of patients based on vascular characteristics for clinical trial enrollment or personalized treatment approaches.

Another object, feature, or advantage is to facilitate longitudinal monitoring of pulmonary vascular changes in response to treatment or disease progression.

It is a still further object, feature, or advantage of the present disclosure to provide biomarkers that correlate with established measures of pulmonary structure and function, including emphysema extent and airflow limitations measured by pulmonary function tests.

Yet another object, feature, or advantage is to support the investigation of the relationship between arterial tree characteristics and hypoxic pulmonary vasoconstriction in areas of inflammation.

It is a further object, feature, or advantage of the present disclosure to enable assessment of arterial-to-airway relationships across various disease states and patient populations.

Another object, feature, or advantage is to provide methods capable of functioning with varying image quality, inflation levels, presence or not of contrast agents, and anatomical variations.

It is a still further object, feature, or advantage of the present disclosure to support the exploration of pulmonary vascular contributions in respiratory disease etiology.

One or more of these and/or other objects, features, or advantages of the present disclosure will become apparent from the specification and claims that follow. No single embodiment need to provide each and every object, feature, or advantage. Different embodiments may have different objects, features, or advantages. Therefore, the present disclosure is not to be limited to or by any objects, features, or advantages stated herein.

### BRIEF DESCRIPTION OF THE DRAWINGS

Illustrated embodiments of the disclosure are described in detail below with reference to the attached drawing figures, which are incorporated by reference herein.

FIG. 1 is a flow diagram illustrating one example of a method.

FIG. 2 is a flow diagram with a more specific example of the method.

FIG. 3 illustrates: Top) 5 lungs of different calibers; Bottom) 24 standard locations on each, with consistent color-coding.

FIG. 4 shows the lungs of the same subject at Left) the TLC volume along with the airways, Right) the FRC volume along with the arteries obtained using AI. Either volume can be used for normalization of the arterial size. Whichever is selected as the comparator should remain consistent across a population for standardization of anatomic characterization. TLC (maximum inspiratory effort) is more repeatable.

FIG. 5 illustrates suggested methods for choosing standard locations on the arterial tree: 1- based on Strahler ordering of the arterial branches alone, 2- based on correspondence with standard airway locations, 3- based on entry location into lung sub-segmental regions.

FIG. 6 illustrates: Top) An example result of the method 1 in the above; Bottom) An example result of the method 2 in the above.

FIGS. 7A and 7B illustrate examples of an analysis pipeline.

FIG. 8 is a snapshot of the manually and semi-automatically curated Training Dataset: 80 Cases. The 3 rows are, 1- coronal slices of lung CT displayed to scale, 2- 3D views of the arterial and venous trees of the same subjects shown in 3D, and 3- only the arterial trees shown within the lungs, all sketched to scale, and from a consistent 3D visualization camera positioned at a consistent distance posterior to the patient.

FIG. 9 illustrates: Graph creation process. Blue dots: computed skeleton, black edges and red nodes: simplified graph representation, green dots: complications going from one node to another, encountering a loop (left), and a lump (right).

FIG. 10: Three methods have been described for establishing standard locations on the arterial tree. FIG. 10 shows the progression from basic Strahler ordering (top) to diameter-defined Strahler ordering (bottom), creating clearly separated orders with non-overlapping diameter ranges. The boxed range (here corresponding to max order (6) – 1 = 5 to max order – 4 = 2) shows the selected standard range here. The range can be altered to include more or less of the central or peripheral orders (segments).

FIG. 11 shows the results for another study, on a separate cohort of patients, using the diameter-defined Strahler ordering to find standard locations. The results are strongly consistent with the results of the artery-airway matching method study (FIGS. 15-16).

FIG. 12 illustrates the suggested pipeline steps. A) 15 slices of the FRC image, slices starting 5 -voxels immediately before and after the lung extent. B) Same for the TLC image. C) Result of the image registration step: image B has been warped and overlaid on top of image A, with a different colormap; any shadows that might appear will signify a registration imperfection. D) The lung mask and the arterial tree segmented using AI from the FRC image. E) The lung mask and airways segmented from the TLC image and labeled. H) The tree of part D shown alongside a minimal graph whose edges have the branch diameter information. I&J) The arterial tree, along with the airway and its graph warped from TLC down to FRC. K) Result of the matching step. F&G: Other views of the arterial tree that might expose enlargement or ballooning.

FIG. 13 illustrates the scatter plot of the orientations product vs distance of all the arterial branches relative to a specific airway branch under question (here RB1), and the ground truth target in red ring, when using only 2 features and a simple hand-crafted rule (maximum alignment, minimum distance) to find the match.

FIG. 14 illustrates examples of some underlying complications in matching airway and arterial trees: A unique matching arterial segment cannot always be found corresponding to a given airway segment. A match might not exist at all (left), or several arterial segments may be fed by (and correspond to) the same singular airway segment (right).

FIG. 15 illustrates: Left) The study population characteristics. Right) Scatter plot of ArtLR using TLC vs FRC volumes as the comparator for normalizing the arterial size.

FIG. 16 illustrates correlations of the arterial dysanapsis index with select lung function or structure biomarkers, in multiple linear regression analyses adjusted for age, sex, and BMI.

FIG. 17 illustrates Left) a dichotomy in the distribution of the male vs female pulmonary arterial diameters, and Right) ArtLR being significantly correlated with and predicted by BMI when adjusted for the above cofounders.

FIG. 18 shows the 110 arterial trees along with the FRC lungs.

FIG. 19 shows the 110 airway trees along with the TLC lungs.

FIG. 20 shows a collage of the 110 arterial trees found in this study.

FIG. 21 shows one subject out of the 110 in the pilot study, along with its demographic and structural and functional biomarkers, and the proposed ArtLR indices, where the subject stands against the others in the cohort, along with its rank, and how the new index generally correlates positively or negatively with the other biomarkers.

FIG. 22 shows samples of an incidental finding using our methodology that depicts enlarged main pulmonary arteries and a ballooning effect which can be easily and automatically flagged using our algorithm, which also warrants further clinical investigation.

FIG. 23 illustrates the potential for easy extension of the above-found arteries and veins into fuller trees extending to the lung peripheries.

FIG. 24 shows one possible AI methodology for the segmentation or the improvement of the segmentation of the arterial and venous trees from medical images. Methods simpler or more complicated than this may be used to fulfil the same purpose and any number of machine learning methods are contemplated.

### DETAILED DESCRIPTION

The present disclosure describes methods and systems for measuring the relative size of the pulmonary arterial tree compared to the lungs. This relationship is quantified as a biomarker called the pulmonary arterial-to-lung ratio (ArtLR), which has significant utility in assessing pulmonary structure and function, particularly in the context of respiratory diseases such as Chronic Obstructive Pulmonary Disease (COPD).

The methods described herein utilize medical imaging data, particularly computed tomography (CT) scans, to segment and characterize pulmonary structures. The key innovation involves the standardized measurement of arterial dimensions at predetermined anatomical locations, with subsequent normalization by lung volume to create a dimensionless index that can be compared across sizes and across individuals robustly.

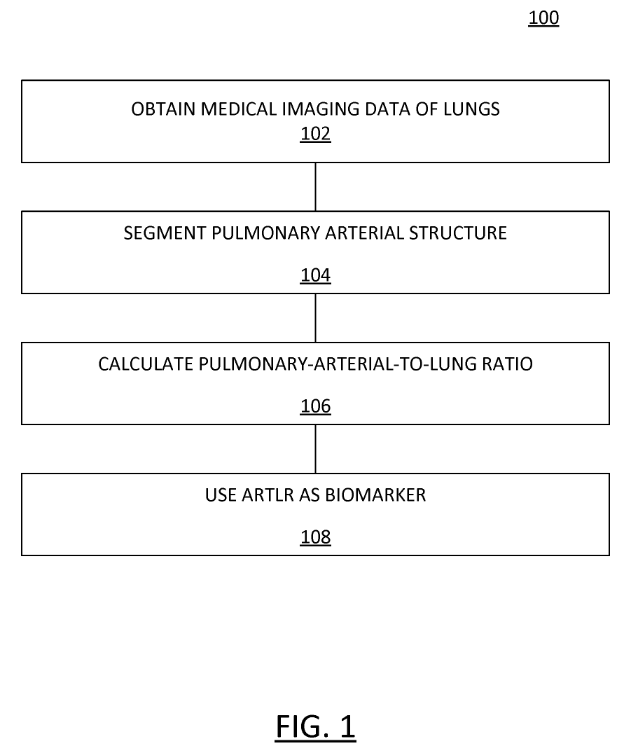

As illustrated in FIG. 1, the method begins with obtaining medical imaging data of a subject's lungs. This imaging data may include contrast-enhanced CT scans, dual-energy CT (DECT) scans, or other modalities capable of visualizing pulmonary structures. From this imaging data, pulmonary arterial structures are segmented using automated or semi-automated techniques, which may include machine learning approaches such as convolutional neural networks or traditional image processing methods, or more recent much more advanced methods like latent diffusion models for segmentation.

Of particular significance is the calculation of the pulmonary arterial-to-lung ratio (ArtLR). This calculation involves determining a relationship between arterial dimensions at predetermined anatomical locations and lung volume. The arterial dimensions may include branch diameter, cross-sectional area, or branch volume. The lung volume may be determined from total lung capacity (TLC) or functional residual capacity (FRC) measurements, or any mathematical functions of those (for example square or cube roots of them, such that the resulting index can become dimensionless).

Various approaches may be employed to identify the predetermined anatomical locations for measurement. An arterial tree-based approach uses structural characteristics of the arterial tree itself, such as branch orders, as in Strahler ordering of the branches, to identify consistent locations across individuals; for example, a consistent target range of orders can be selected as the consistent standard target locations. An airway-guided approach identifies arterial segments corresponding to standard locations on the airway tree, establishing an anatomical correspondence between these closely-following branching structures. A regional approach uses anatomical partitioning of the lungs into segments or lobes and identifies arterial branches at sites corresponding to these regions.

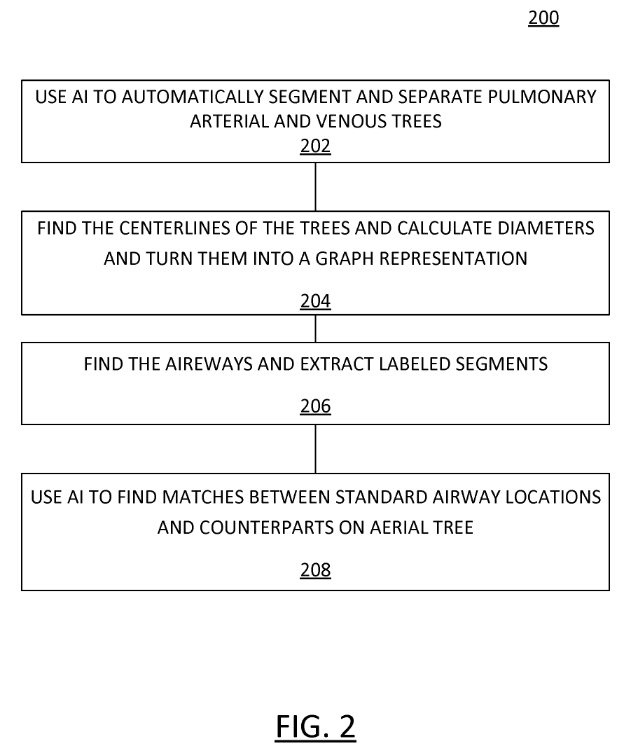

FIG. 2 illustrates a multi-stage implementation of the method, comprising segmentation of pulmonary structures, including arterial, venous, and airway trees; structural abstraction of these trees into centerline-based graph representations that also carry diameter or volume information; identification of standard anatomical locations for measurement; and computation of the ArtLR index based on measurements at these locations.

The calculated ArtLR serves as a biomarker for assessing pulmonary structure and function. This biomarker may be used independently or in combination with other clinical and imaging parameters to evaluate disease risk, progression, or response to treatment. Significant correlations have been observed between ArtLR and established measures such as emphysema extent, airway morphology, and pulmonary function tests.

The methods described herein may be implemented in software, hardware, or a combination thereof. They may be integrated into clinical workflows, research protocols, or diagnostic systems to provide automated or semi-automated assessment of pulmonary vascular characteristics.

In the following sections, specific implementations of these methods are described in detail, including techniques for arterial tree segmentation, approaches for standardizing measurements, algorithms for establishing anatomical correspondences, and validation of the resulting biomarker against clinical parameters.

FIG. 1 illustrates one example of a method 100 which may be used for assessing pulmonary vascular characteristics. In step 102, medical imaging data of a subject’s lungs is obtained. In step 104, the pulmonary arterial structure is segmented. In step 106, a pulmonary arterial-to-lung ratio (ArtLR) is calculated by determining a relationship between arterial dimensions at predetermined anatomical locations and lung volume. The predetermined anatomical locations may include a subset of standard airway segment labels such as an RB1 label and an RB4 label. The arterial dimensions may include at least one of branch diameter or branch volume as measured at the predetermined anatomical locations. The lung volume may be determined from total lung capacity (TLC) or functional residual capacity (FRC) obtained from the medical imaging data. Whichever is selected as the comparator should remain consistent across a population for standardization of anatomic characterization. TLC (maximum inspiratory effort) is more repeatable. Arterial size is best assessed at FRC whereby the parenchymal-arterial mechanical interactions do not serve to stretch or compress the vascular geometry. However, airways are best detected at TLC where bronchial segments are detected further out to the lung periphery.

It should be understood that imaging of the lung at the resting inflation volume or FRC is used for obtaining the arterial tree as the more common total lung capacity (TLC) inflation level will distort the parenchyma and compress vascular geometry at the higher inflation level. However, the comparator volume (by which arterial diameters are normalized) may be the FRC or TLC. However, it may be advantageous to use TLC as it appears more reliable and repeatable. To obtain the airways, however, we preferably use the TLC volume, where the airways are more prominent and distinct, enabling easier, more accurate and more extensive segmentation and labeling of the airways, and then warping those airways and labels down to the FRC volume using image registration and transformation. This allows for establishing reliable airway segmentations and labels.

In step 108 the calculated ArtLR may be used as a biomarker for assessing pulmonary structure and function. The method may further include segmenting a pulmonary venous tree and an airway tree from the medical imaging data and automatically matching tree segments to the predetermined airway anatomical locations based on metrics of geometric similarity, including proximity and orientation. The automatic matching may be performed using a trained machine learning model.

In one embodiment, standard locations may be found on the arterial tree which only depend upon the arterial tree itself not needing, for example, correspondence with airways. For example, Strahler ordering of the branches may be used starting at order 1 at the terminals and upping the orders as similar orders join and once all branches have been ordered, selecting a consistent range of orders as the standard locations. Alternatively, standard locations may be defined as those corresponding to standard locations on the airways such as labeled segments.

According to one aspect, the methodology of the present disclosure has four stages. This multi-stage method is provided for determining a physiological index that characterizes pulmonary arterial structure. This index, referred to herein as the arterial-to-lung ratio (ArtLR), is computed by analyzing vascular features at standardized anatomical locations and normalizing those measurements by lung volume. The method includes four primary stages: segmentation, structural abstraction, anatomical correspondence, and index computation.

FIG. 2 illustrates the four stages. The first stage 202 uses AI to automatically segment and separate the pulmonary arterial and venous trees. The second stage 204 finds the centerlines of the trees and calculates their diameters and turns them into a graph representation. The third stage 206 finds the airways, and extracts labeled segments (such as RB1, RB4, RB10, LB1, …) using software such as the VIDA Apollo software. The last stage 208 uses AI to find matches between the standard airway locations and their counterparts on the arterial tree.

In the first stage, medical imaging data of a subject’s lungs—such as contrast-enhanced computed tomography (CT) scans—is processed to segment anatomical structures of interest. Specifically, the pulmonary arterial tree, the pulmonary venous tree, and the airway tree are each segmented from the image data. The segmentation is performed using an automated process, which may include application of trained machine learning models. Suitable models may include convolutional neural networks, U-Net architectures, latent diffusion models, or other deep learning-based segmentation frameworks. These models may be trained using manually or semi-automatically segmented vascular and airway trees curated from a training dataset, thereby enabling high-fidelity separation of arterial and venous structures, even in regions where close spatial proximity complicates conventional segmentation methods.

In the second stage, the segmented pulmonary arterial tree is further processed to extract centerlines, which define the geometric paths of vascular branches throughout the lung. From these centerlines, a graph representation of the arterial tree is generated. This representation consists of nodes and edges, where nodes correspond to bifurcation points or endpoints within the vascular structure, and edges represent individual vessel segments. Each edge in the graph is associated with one or more geometric features, including but not limited to diameter, length, and orientation. This graph-based abstraction provides a topologically consistent and computationally efficient representation of the vascular tree suitable for anatomical comparison, physiological modeling, and machine learning applications.

The third stage involves identification and labeling of anatomical locations on the airway tree. A plurality of standard airway locations is identified within the segmented airway tree, and each location is assigned an anatomical label according to a predefined reference scheme. These airway labels may include standardized bronchopulmonary segment identifiers such as RB1, RB4, RB10, LB1, and similar region-specific notations. In certain embodiments, software tools such as VIDA Apollo may be used to assist in labeling and standardizing these locations across subjects. For a subset of these labeled airway locations, corresponding arterial segments are identified within the pulmonary arterial tree. The correspondence is established based on one or more anatomical or geometric features, such as proximity, relative orientation, similarity in branch diameters or lengths, or generational position on the trees. In one embodiment, a trained neural network is applied to evaluate a multi-dimensional feature vector comprising proximity, orientation, diameters ratio, generational position, and a unique airway segment identifier to enable treating different segments differently, say trachea vs RB4. Not all airway locations yield reliable arterial matches; therefore, a reliability criterion is used to select a subset of airway locations—such as nineteen of the original set—for further analysis, thereby increasing the robustness of the computed index.

In the fourth and final stage, the physiological index is computed. For each matched arterial segment, one or more size-related measurements—such as the average diameter or cumulative volume—are obtained. These measurements are aggregated across the subset of reliable standard locations to produce a composite arterial size metric. This metric is then normalized using a measure of lung volume, which may be derived from the same imaging data used in the segmentation step. Lung volume may be measured using total lung capacity (TLC) or functional residual capacity (FRC), and the normalization may involve either direct division by lung volume or by the cube root of lung volume, depending on the desired form of the index. The resulting value, the ArtLR, reflects the proportional relationship between arterial size and lung volume, thereby enabling assessment of pulmonary arterial dysanapsis and potential structural mismatches relevant to pulmonary health.

The physiological index thus computed may be used independently or in combination with other functional, structural, or clinical biomarkers to assess the likelihood of pulmonary disease or dysfunction, such as chronic obstructive pulmonary disease (COPD). In some embodiments, statistical correlations are performed between the ArtLR and imaging-derived or physiologic parameters, such as emphysema extent, parenchymal textures (like AMFM textures), or lung function measures, to evaluate disease phenotype or progression.

### Pulmonary Arterial Tree Standardization Using Strahler Ordering Approach

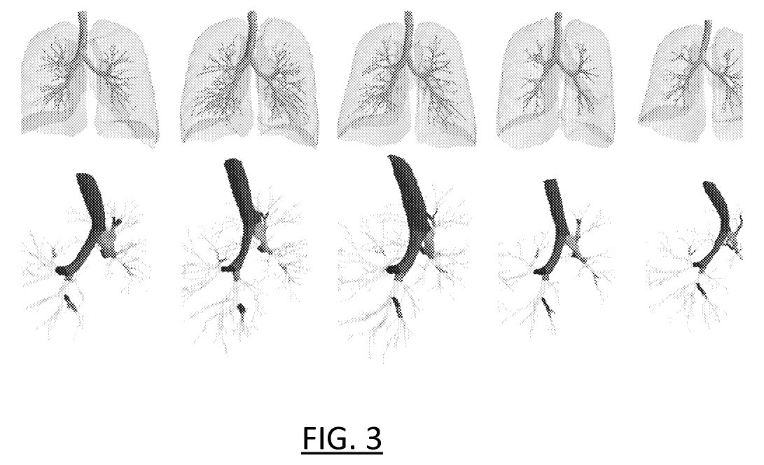

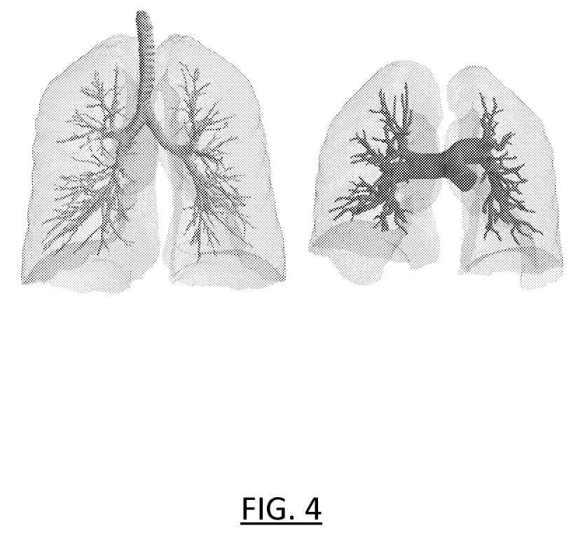

A first study was performed as shown beginning at FIG. 3. That study involves finding standard locations on the arterial tree which depended only on the arterial tree itself, not needing correspondence with airways. FIG. 3 illustrates the lungs with airways of different calibers as well as the 24 standard locations.

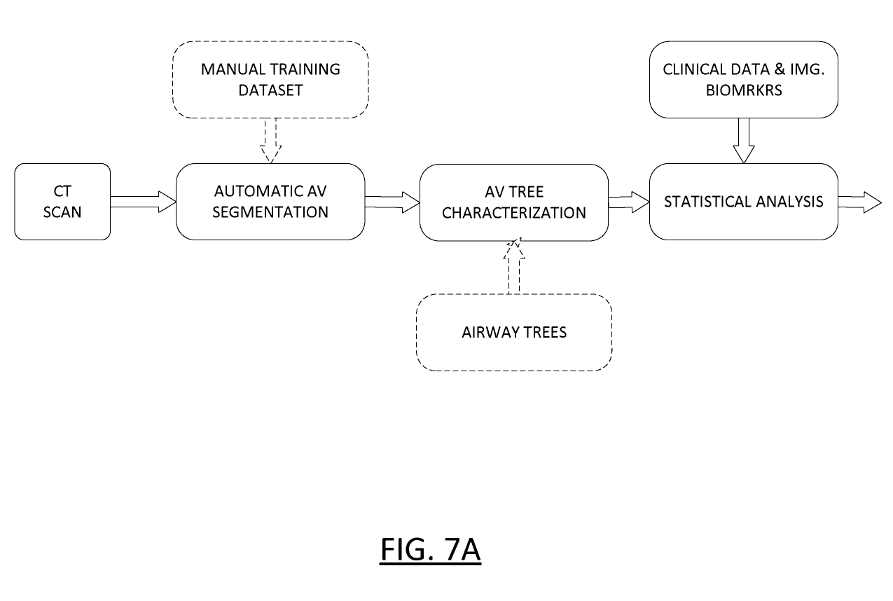

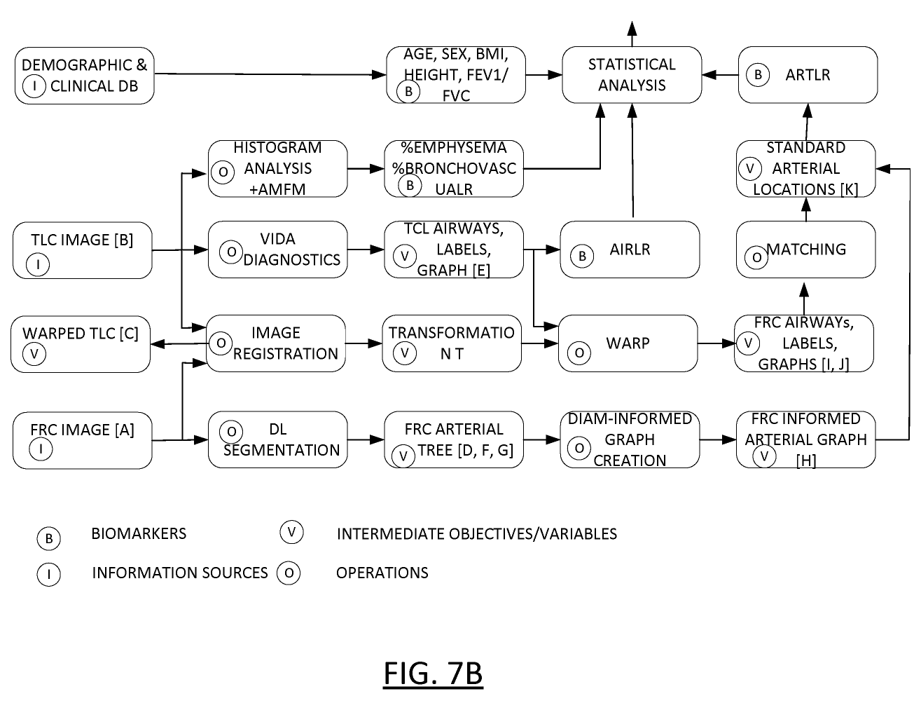

A semi-automated segmentation of arterial and pulmonary trees was performed using the pipeline of FIGS. 7A and 7B. The pipeline includes automatic segmentation of combined pulmonary arteries and veins, automatic characterization of the trees to find standard locations for use in defining ArtLR and performing statistical analyses against other imaging and non-imaging-based biomarkers.

As shown in FIGS. 7A and 7B, contrast-enhanced dual-energy CT (DECT) scans at functional residual capacity (FRC) have been used to form a training set for AI applications. To enable automatic segmentation of the vascular trees, a dataset containing 80 manually or semi-automatically segmented vessels was created. This set of segmentations was used to train the automated methods.

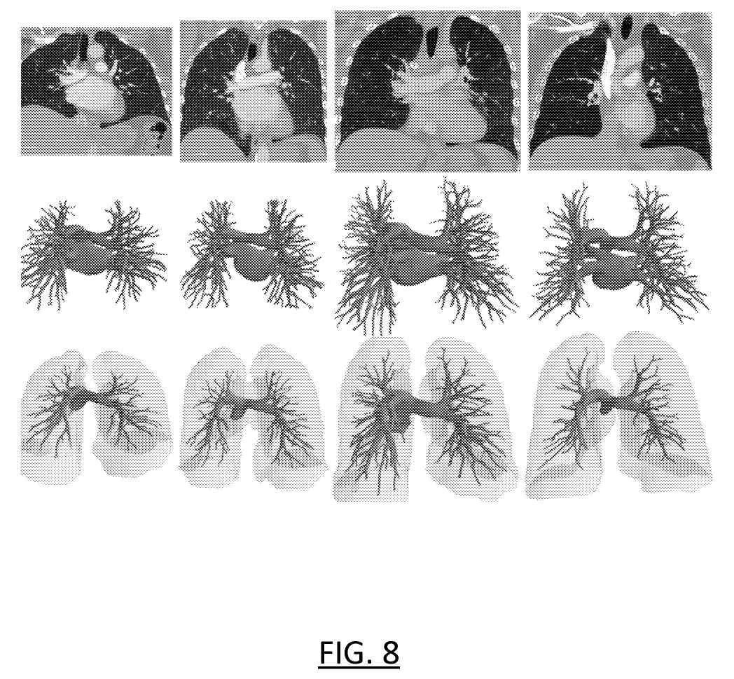

FIG. 8 is illustrative of a portion of the training dataset snapshot. As shown, there are 3 rows which include lung snapshots displayed to scale, 3D views of the arterial and venous trees, and the arterial trees within the lungs all drawn to scale and from a consistent 3D visualization camera positioned at a consistent distance posterior to the patient.

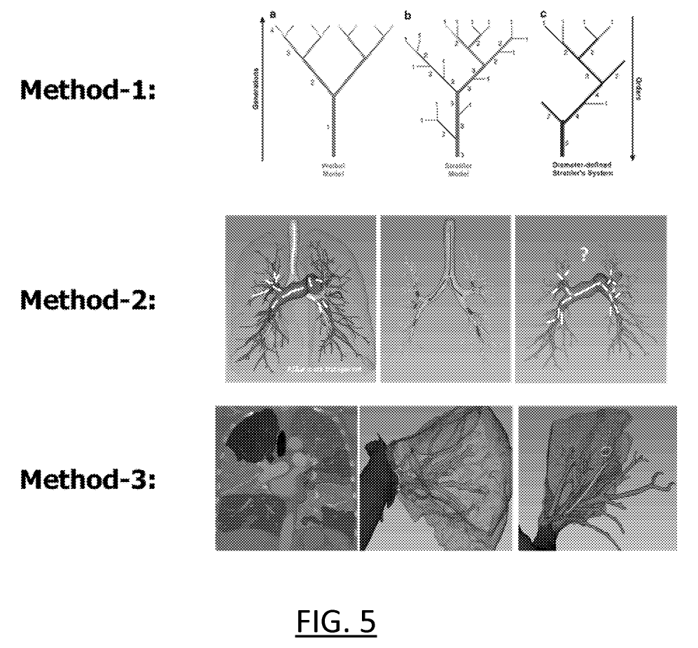

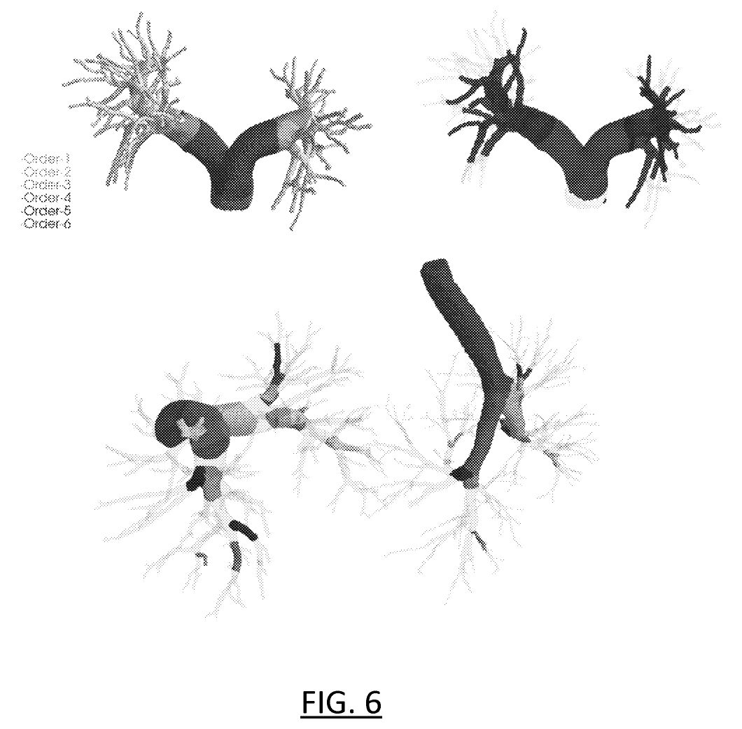

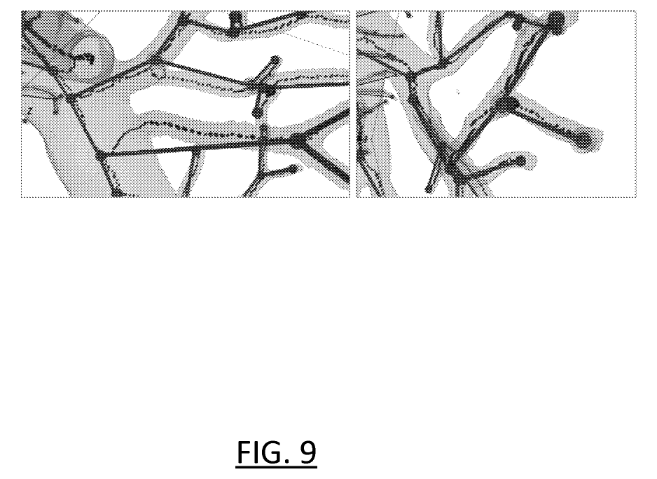

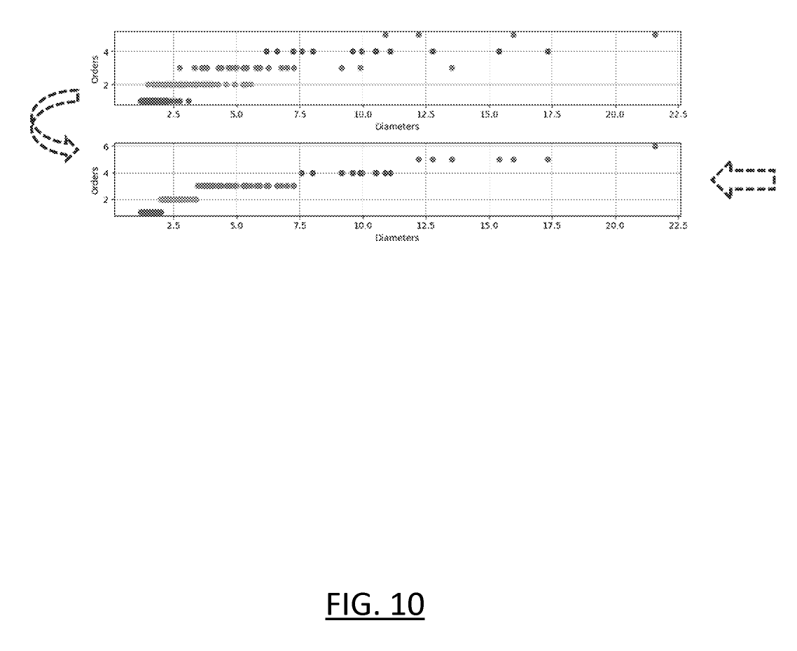

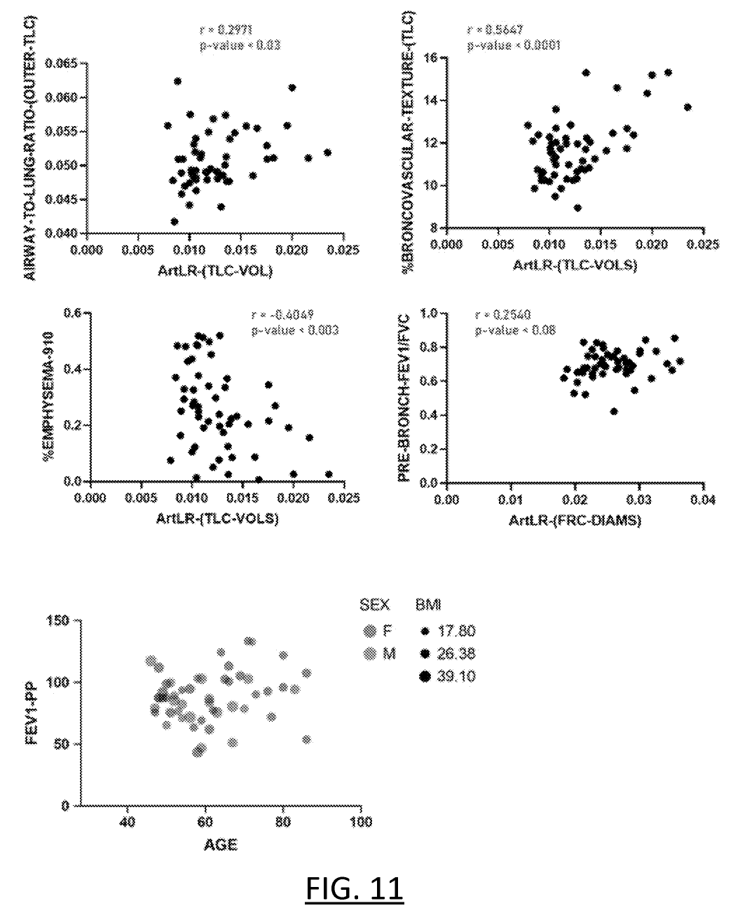

Tree characterization was performed. To do so, after obtaining the 3D arterial masks, the arterial tree centerlines are then computed and converted into graph representations. FIG. 9 is representative. Then standard locations were determined. Various methods may be used for identifying standard locations on the trees consistent across different subjects. In a first method based on the arterial tree alone, a diameter-defined Strahler ordering of the branches and selection of branches of a particular order range as the targets is performed. According to another method one may start with the 24 standard airway locations (or a subset thereof) used to define AirLR and then identifying their arterial segment counterparts. A third method may be based upon the branching patterns within individual sub-lobar segmental regions. FIG. 9 illustrates the graph creation process. FIG. 5 illustrates suggest methods for choosing standard locations. FIG. 6 illustrates example results for two of the methods.

A number of different matching strategies may be used such as matching full trees versus matching individual branches, or hand-crafting feature vectors / decision rules versus learning them automatically (for example by feeding a full geometry-informed graph into a Graph Neural Network to learn both automatically).

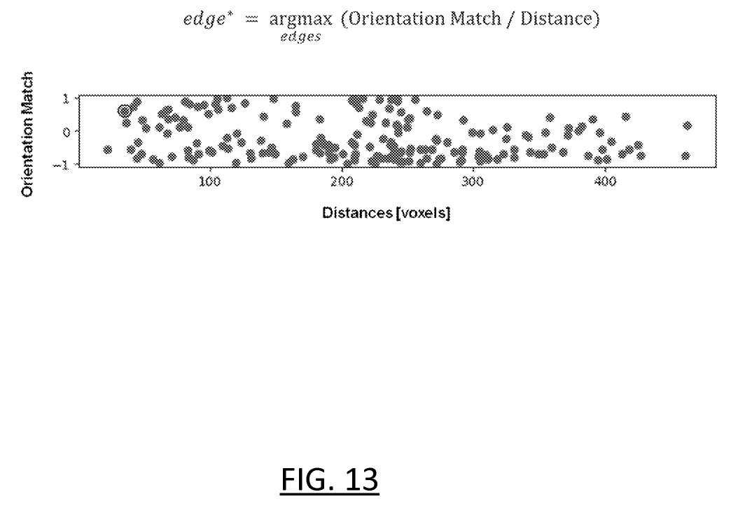

FIG. 13 is an example which shows the case with 2 hand-crafted features, and a simple hand-crafted decision rule (least distance, highest orientations product), which although useful does not handle all matching situations. The scatter plot shows the orientations product versus distance of all the arterial branches relative to a specific airway branch (here RB1), and the ground truth target (left, upper point), when using only 2 features to find the match.

A more complex model with individual branch matching may be used. For example a 6-dimensional features vector and a learned decision rule using a neural network may be used. The features may be based on, without limitation, proximity of the airway/vessel branches, orientations, generational positions, ratio of branch lengths, ratio of branch diameters, and the airway segmentation code (e.g. RB1, RB4, …). For a variable length no-order sequence of multi-dimensional (2-D, 6D, …) points, a “best” match should be selected for each airway segment. One method of doing so is to treat the points as a sequence and use a Long-Short-Term-Memory (LSTM) neural network architecture. Any similar method to “pick” the best element in a multi-dimensional features vector can be used.

A training dataset of 6 cases was created with 24 standard airway locations each and their corresponding ground truth arterial locations (an overall 144 scenarios). An accuracy of around 95 percent in finding the right match is recorded using a 6-fold cross validation scheme.

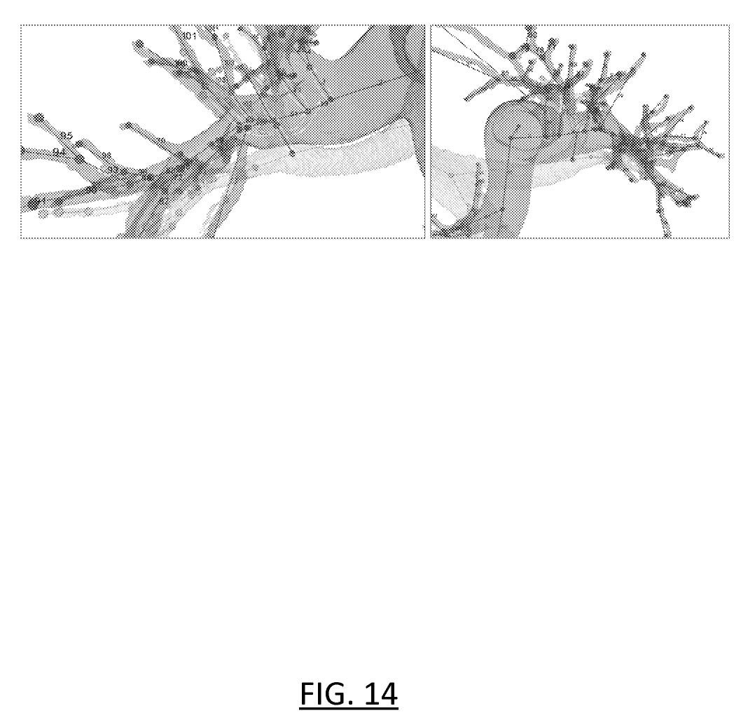

FIG. 14 illustrates examples of some underlying complications. For example, a unique matching arterial segment cannot always be found corresponding to a given airway support. A match might not exist at all (left) or several arterial segments may be fed by (and correspond to) the same singular airway segment (right). Locations particularly susceptible to such events have been identified and can be excluded from the standard measurements, for example segments closest to the left upper lobe (LUL).

A number of variations are contemplated. For example, upon selection of the target branches, the average branch diameters or sum of branch volumes may be divided by the cube root of the lung volume or the lung volume, respectively. As previously explained, there is the choice of using FRC or TLC as lung volume, thus providing four variations to define ArtLR.

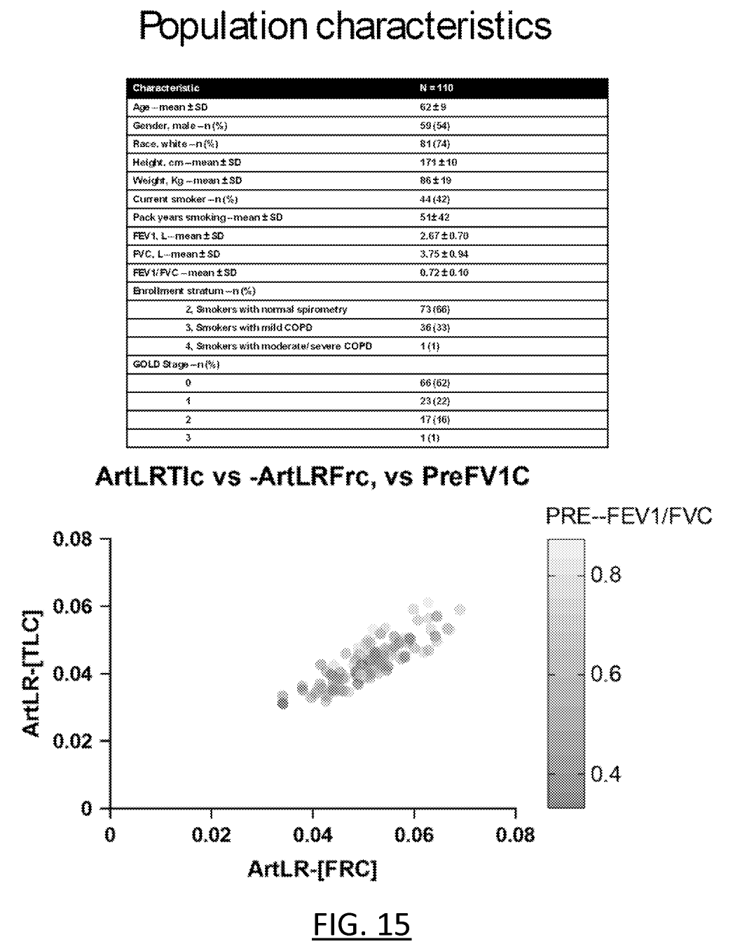

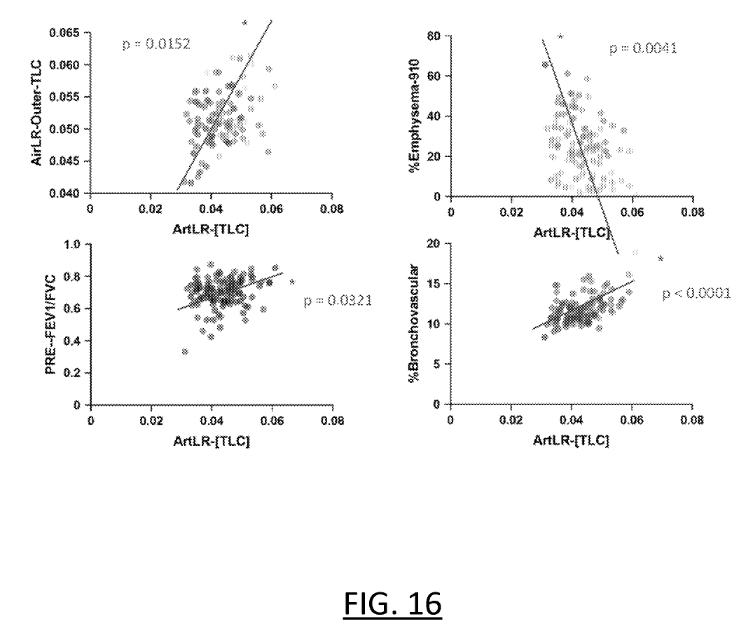

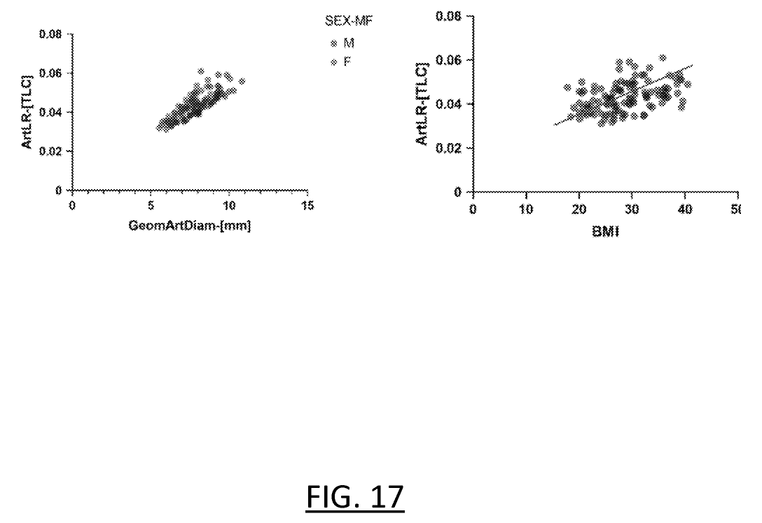

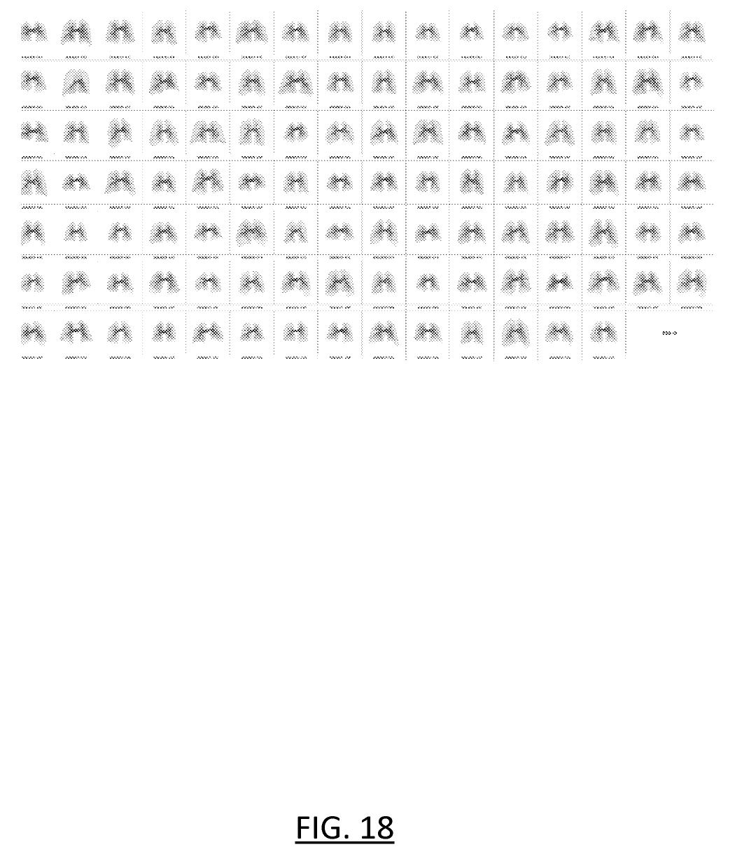

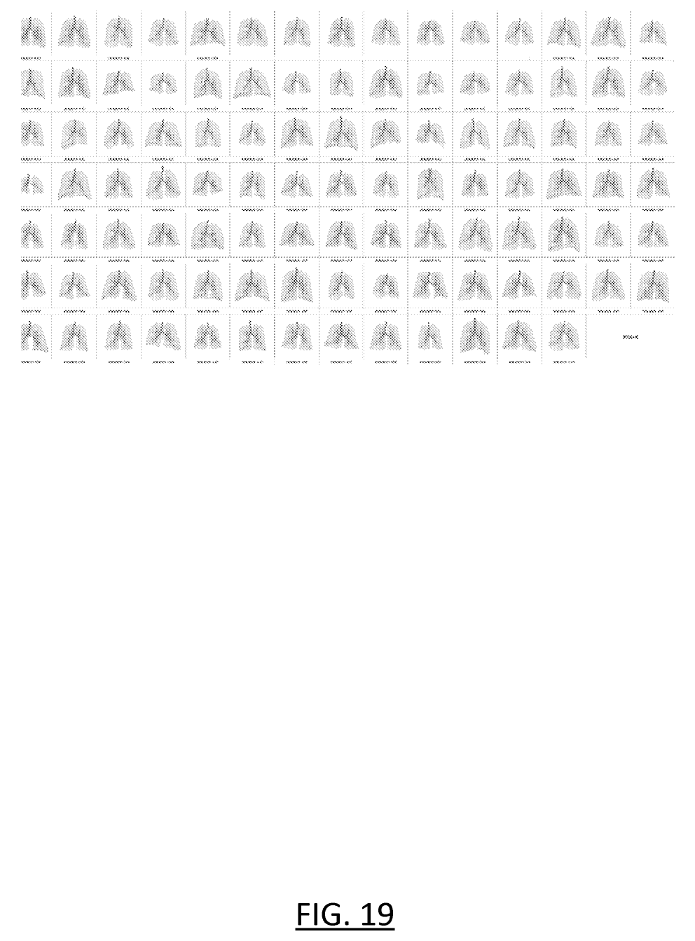

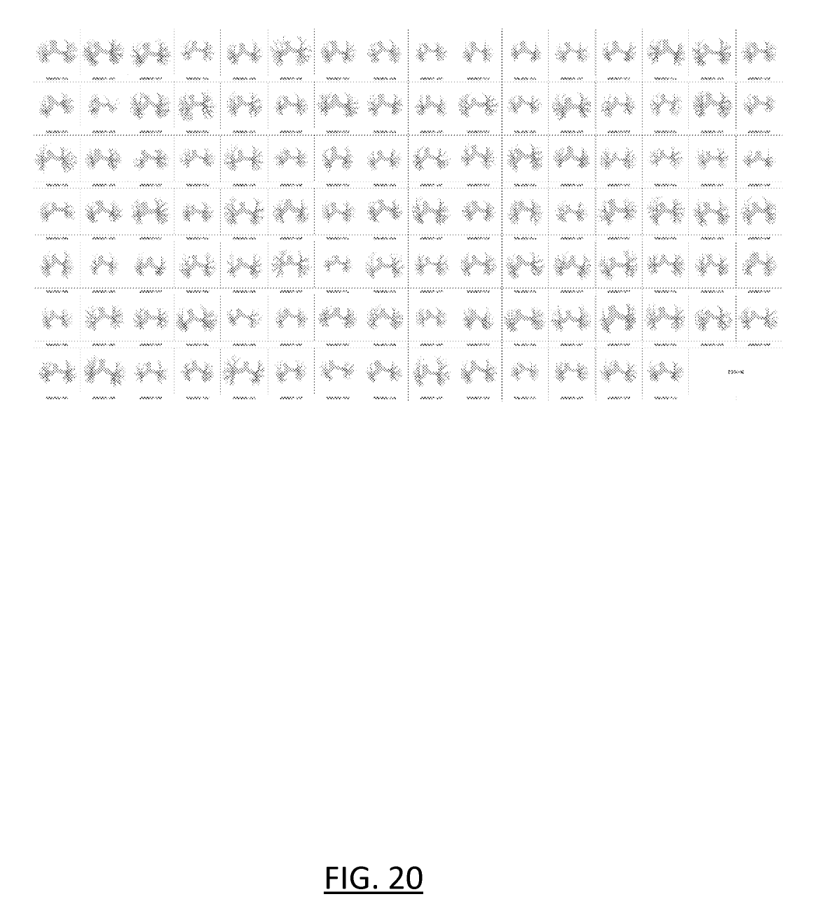

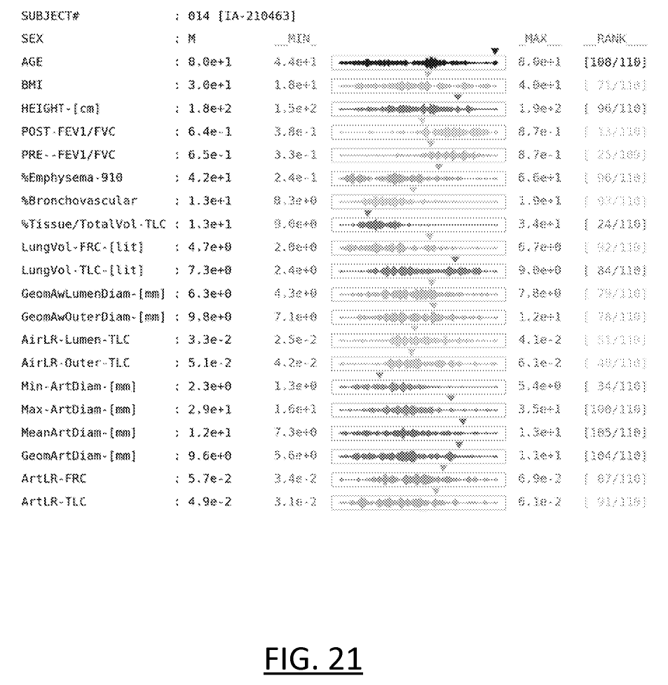

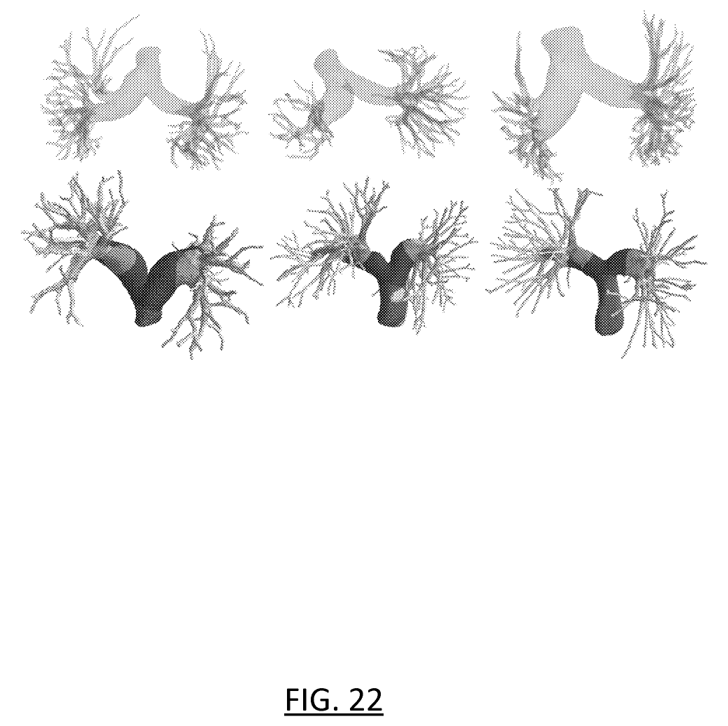

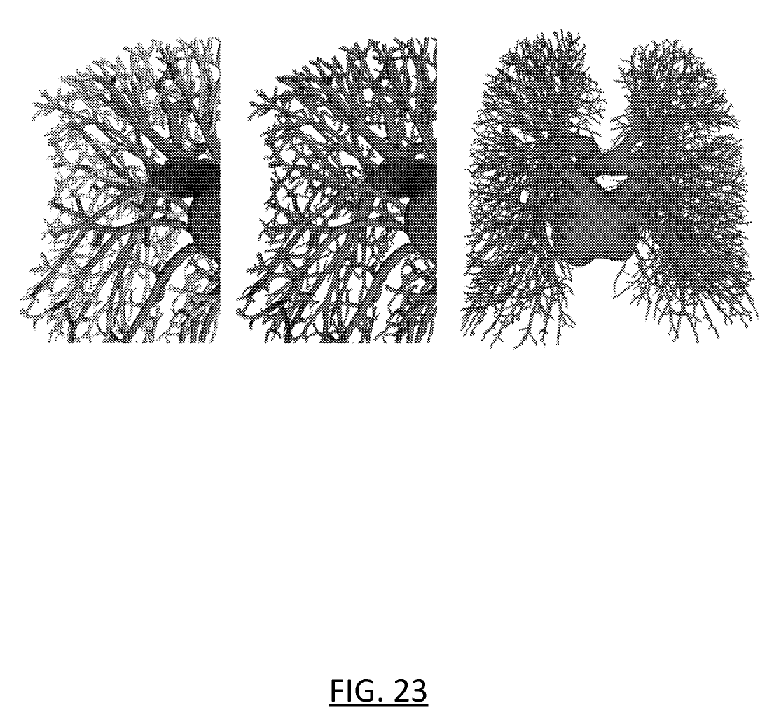

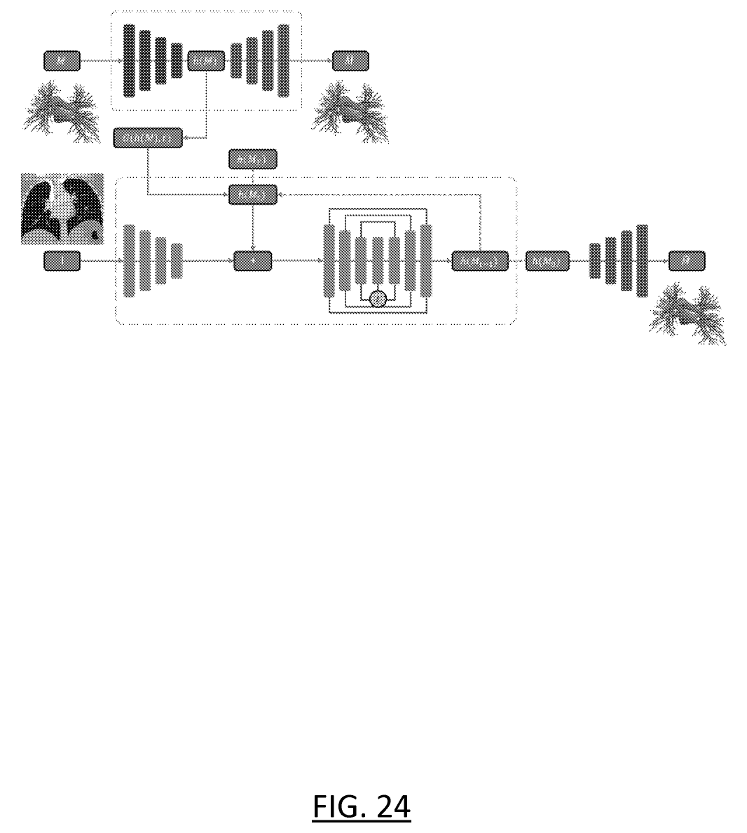

The results of a first study using the Strahler method are using the 54 existing in the ground-truth segmentation dataset, together with two variations of the method has been used to investigate the ArtLR (the arterial dysanapsis index). To obtain the results, multivariate linear regression was performed to test the association of ArtLR with functional and structural biomarkers. All multivariate models were adjusted for age, sex, and BMI. Using a variation of the method using TLC and branch volume, in adjusted analyses, a 1-SD increment in ArtLR was associated with 6.65 unit decrease in %Emphysema-910 (95%CI: 2.78 to 10.52; p = 0.001), 0.9187 unit increase in %Broncovascular-Texture (95%CI: 0.5906 to 1.247; p < 0.0001), 0.31 SD increase in Airway-to-Lung Ratio (95%CI: 0.03 SD to 0.60 SD; p = 0.034). In the method using FRC and branch diameter, a 1-SD increment in ArtLR was associated with a 0.028 unit increase in PreBronch-FEV1/FVC (95%CI: 0.002 to 0.053; p = 0.035). FIG. 15 illustrates demographics of the study population and correlations of the arterial dysanapsis indices with select lung function or structural biomarkers.

In the above example, a training set was established for the development of a deep learning-based algorithm to separate pulmonary arterial and venous trees using CT images. Using TLC lung volume as the comparator, we have shown that the Strahler-based arterial volume as the arterial dysanapsis index has a significant relationship with AirLR, texture-based broncho-vascular volume (AMFM method for texture assessment) and emphysema volume as shown in the statistics above. Using FRC lung volume as the comparator, we have shown that the Strahler-based average arterial diameter significantly predicts pre-broncho dilator FEV1/FVC ratio. With this newly developed image-based assessment of the pulmonary arterial tree, we provide a means of comparing the relative significance of airway vs vascular influences and responses associated with pulmonary pathophysiology.

In addition, any number of different deep neural network architectures may be used for automatic segmentation of the AV trees. For example, a U-Net model may be used, a 3D Latent Diffusion Model (LDM) or other type of model.

This study paves the way for the introduction of a new biomarker representing pulmonary arterial anatomy that captures the size mismatch between the pulmonary arterial tree and the lung and/or establishes a method for indexing peripheral pulmonary vascular resistance. Our ability to automate the extraction of the pulmonary arterial tree will provide the ability to explore the arterial role in emphysema etiology within much larger ongoing multi-center studies.

A database of 80 semi-automatically segmented pulmonary arterial and venous trees was created from dual-energy contrast-enhanced CT scans taken at FRC (Functional Residual Capacity), at an upsampled isotropic resolution of 0.38 mm. The segmentations include the trees from the pulmonary trunk, midway into the lung periphery, using a consistent watershed segmentation at 0.6 mm scale, and quality controlled by 2 observers including a board certified radiologist. Clinical and demographic data along with quantitative disease measures obtained included (but not limited to) texture-based (AMFM) measures of Bronchovascular Prominence and %emphysema along with spirometry including FEV1 %predicted. For each 3D arterial tree, the centerline was extracted and turned into a minimal graph with only terminal/branching/root nodes, and the volume and average diameters were calculated for each branch. A diameter-defined Strahler analysis was done, considering diameters when assigning the orders, from the smallest at the terminals to the largest at the root. A larger lung might end up with a higher order at the root compared to a smaller lung, but picking the target orders relative to the root (say max order - 1) will ensure that the same consistent anatomical levels are picked regardless of subject size, etc.

### Automated Pulmonary Arterial Tree Segmentation and Matching Using Depp Learning

A second study was performed to further validate and refine the methods described above. This second study provides additional technical approaches for segmentation, standardization, and validation of the ArtLR biomarker.

A deep learning approach was employed for the segmentation and separation of pulmonary arterial and venous trees. A dataset containing 80 manually or semi-automatically segmented vessels was created to train the automated methods, as illustrated in FIG. 8. The segmentations were performed on contrast-enhanced dual-energy CT (DECT) scans at functional residual capacity (FRC).

A standard UNet convolutional neural network architecture was implemented to learn the segmentation task. The network was trained to classify each voxel in the input 3D CT image into one of three classes: background, artery, or vein. The output of the network was a 3D image of the same size as the input image, with voxel values of 0, 1, or 2 corresponding to the three classes, respectively. The segmented arterial trees were then extracted for further processing.

After obtaining the 3D arterial masks, the arterial tree centerlines were computed and converted into graph representations. This conversion process included skeletonization, initial graph representation, and pruning until the graph became a cycle-free tree in the graph theoretical sense.

Diameter information was added to each skeleton voxel, calculated as twice the shortest distance to the background. For each branch, an average diameter was determined by averaging values from the middle third points along the branch. The resulting graph representation comprised a root, branching points, and terminal points, with edges connecting them in a tree structure. Each edge was also annotated with average diameter information.

To establish correspondence between the airway tree and arterial tree, a registration process was employed. Since the airway tree and its standard locations were available in total lung capacity (TLC) scans while the arterial tree was segmented from FRC scans, a registration step was necessary to align these structures. Also, because airways are much more expanded and more easily and robustly segmented in the full inflation level as compared to FRC. But the arteries were obtained from the FRC image as previously explained.

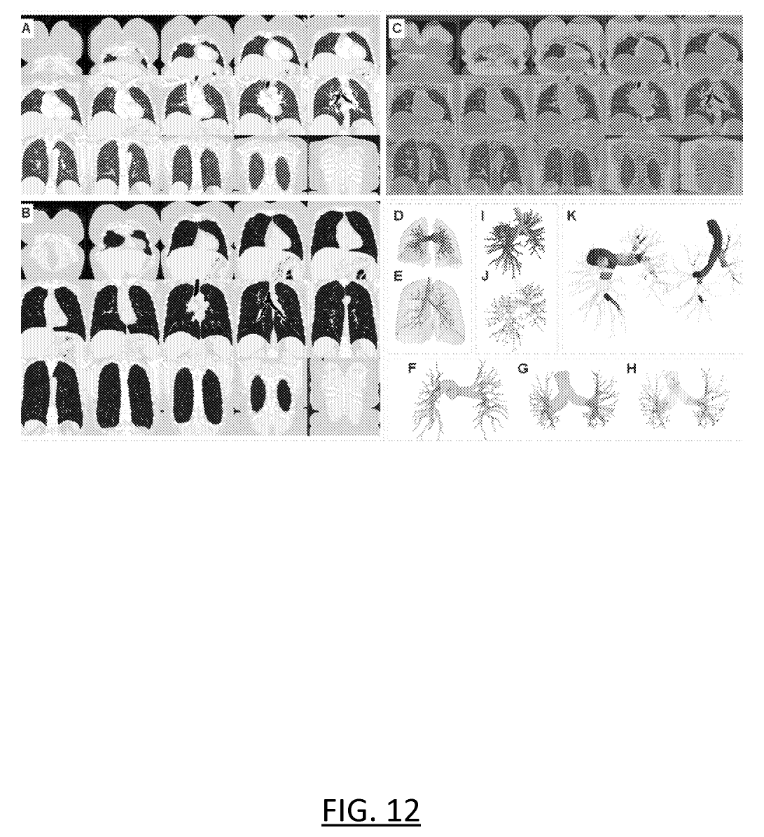

The TLC images were warped to match the FRC images, and the resulting transformation was applied to warp the airways and labels from TLC to FRC space. FIG. 12 shows examples of registration results, with the FRC image shown in grayscale and the warped TLC image overlaid in a color map.

Three methods were identified for establishing standard locations on the arterial tree, as illustrated in FIG. 5, as explained before.

In the current implementation, Method-2 was primarily employed, with the matching algorithm described below.

To match airway locations with their corresponding arterial segments, a machine learning approach using Long Short-Term Memory (LSTM) neural networks was implemented. The matching algorithm utilized a 6-dimensional feature vector based on proximity of the airway/vessel branches, relative orientations, generational positions in their respective trees, ratio of branch lengths, ratio of branch diameters, and the airway segment identifier (e.g., RB1, RB4, etc.).

The algorithm treats the candidate arterial branches as a sequence and produces scores indicating match probabilities. As previously described, FIG. 13 illustrates a simplified version of this matching process, showing a scatter plot of orientations product versus distance for potential matches, with the ground truth target highlighted.

The ArtLR was calculated using the diameters of arterial segments at the identified standard locations. Following the approach used for AirLR, the sub-segmental branches (e.g., RB1a, RB1b, RB4a, RB4b) were first averaged, and then the geometric mean of the resulting diameters was calculated and divided by the cube root of the lung volume. Any subset of the original 24 airway locations, including or excluding the sub-segmentals referred to above, could be used, and it has in fact been shown that the exact selection does not matter very significantly. Both FRC and TLC were evaluated as potential normalizing volumes, providing two variations of the ArtLR biomarker.

The ArtLR biomarker was validated in a cohort of 110 subjects from the SPIROMICS study. Multivariate linear regression was used to test associations between ArtLR and various functional and structural biomarkers, with adjustment for age, sex, and BMI.

The results demonstrated significant correlations between ArtLR and clinically relevant parameters:
A 1-SD decrement in ArtLR-TLC was associated with a 4.198 unit increase in %Emphysema-910 (95%CI: 1.360-7.037; p = 0.0041)
A 1-SD decrement in ArtLR-TLC was associated with a 0.8198 unit decrease in %Bronchovascular texture prominence (95%CI: 0.5037-1.136; p < 0.0001)
A 1-SD increment in ArtLR-TLC was associated with a 0.2515 SD increase in AirLR-Outer (95%CI: 0.04947SD-0.4536SD; p = 0.0152)
A 1-SD increment in ArtLR-TLC was associated with a 0.02083 unit increase in PreBronch-FEV1/FVC (95%CI: 0.001814-0.03984; p = 0.0321)

Additionally, sex and BMI showed significant associations with ArtLR-TLC, where female sex was associated with a 1.068 SD decrease in ArtLR, and a unit increase in BMI was associated with a 0.05788 SD increase in ArtLR (both p-values < 0.0001). Age was not significantly associated with ArtLR. These results very well follow the trends of those seen in the first study using the Strahler method above.

These results indicate that the ArtLR biomarker successfully captures clinically relevant structural and functional characteristics of the pulmonary vasculature and may provide complementary information to existing biomarkers such as AirLR.

Several technical challenges were encountered and addressed during implementation. These include segmentation challenges, registration variability challenges, anatomical variability challenges, and lung inflation differences. With respect to segmentation challenges, structures passing near the iodinated superior vena cava were sometimes not properly segmented. Additionally, in subjects with severe emphysema or significant motion artifacts, segmentation quality could be compromised. These cases were identified through visual inspection and quality control. With respect to registration variability, the quality of registration between TLC and FRC scans varied among subjects. However, the method proved robust to registration imperfections as long as the airways were reasonably aligned. The registration quality was categorized for each subject, ranging from perfect to failed, with most cases achieving acceptable alignment. With respect to anatomical variability, significant variations in arterial tree morphology were observed across subjects, including differences in main pulmonary artery configuration, branch patterns, and tortuosity. These variations were successfully tackled by the matching algorithm, which considered multiple geometric features rather than relying solely on spatial proximity. With respect to lung inflation differences, variability in inflation levels between FRC and TLC scans impacted calculations. In some cases, FRC volumes appeared closer to residual volume (RV), while in others, they approached TLC. These differences were accounted for by evaluating both FRC and TLC as normalization volumes.

### Clinical and Pharmaceutical Applications

The ArtLR biomarker described herein has several potential applications in clinical practice and pharmaceutical development. In clinical settings, ArtLR may serve as a diagnostic tool to identify patients at risk for COPD or other pulmonary disorders characterized by vascular remodeling. The significant correlations observed between ArtLR and established clinical parameters such as FEV1/FVC ratio, emphysema extent, and broncho-vascular texture suggest that this biomarker captures physiologically relevant information about the pulmonary vasculature and its relationship to lung parenchyma.

Clinicians may use ArtLR measurements to phenotype patients with respiratory symptoms, potentially identifying those with primary vascular abnormalities versus those with predominant airway disease. This distinction may guide therapeutic decisions, as different phenotypes may respond differently to bronchodilators, vasodilators, or anti-inflammatory agents. Furthermore, longitudinal monitoring of ArtLR could help assess disease progression or response to treatment, providing an objective measure of pulmonary vascular status over time.

In pharmaceutical development, ArtLR may be valuable for patient selection in clinical trials. For example, clinical trials targeting therapies for pulmonary hypertension secondary to COPD might benefit from enrolling patients with abnormal ArtLR values, as these individuals may represent a subgroup with more pronounced vascular involvement. Conversely, trials of bronchodilators might focus on patients with normal ArtLR but abnormal airway measurements, targeting the airway-predominant phenotype.

The ArtLR biomarker might also serve as a surrogate endpoint in clinical trials, potentially allowing for shorter studies or smaller sample sizes if changes in ArtLR precede or correlate with changes in clinical outcomes. Thus, ArtLR may potentially be used as a predictor of clinically relevant endpoints such as exacerbation frequency, hospitalization, or mortality.

The automated nature of the ArtLR measurement methods described herein facilitates implementation in large-scale studies and clinical practice. The ability to process large numbers of CT scans consistently and efficiently makes this biomarker suitable for population-based studies, multi-center clinical trials, and routine clinical use in centers with appropriate imaging capabilities.

It should also be understood that the biomarker described herein may be incorporated into a variety of artificial intelligence platforms and clinical workflow solutions. The ArtLR biomarker may be implemented as a standalone application or integrated into existing medical imaging analysis suites such as those provided by various vendors in the healthcare AI marketplace. These implementations may include cloud-based platforms that allow healthcare providers to submit CT images for analysis through secure APIs or web interfaces, with results returned for integration into electronic health record systems or radiological reporting workflows.

The biomarker may be particularly valuable when incorporated into comprehensive AI suites and platforms (such as those offered by companies like Blackford, Flywheel, Arterys, or similar vendors) that provide orchestration capabilities, allowing users to send their images to multiple AI applications developed by different companies, all hosted on a unified platform. Such integration enables efficient clinical workflows where ArtLR analysis can be performed alongside other quantitative imaging biomarkers, creating comprehensive pulmonary assessment reports.

Furthermore, the biomarker may serve valuable functions in population health management and risk stratification. Insurance companies, accountable care organizations, and healthcare systems may utilize the ArtLR biomarker as an aid in predicting patient trajectories and outcomes. For example, the biomarker could inform risk models for COPD development, progression, or exacerbation, potentially identifying patients who would benefit from earlier intervention or more intensive management. This predictive capability may support value-based care initiatives by enabling more precise resource allocation and personalized treatment planning.

In pharmaceutical research and development, the biomarker may be implemented in clinical trial management systems to support patient selection, stratification, and treatment response assessment. The quantitative nature of the biomarker makes it particularly suitable for objective longitudinal monitoring, potentially serving as a surrogate endpoint in trials of therapies targeting pulmonary vascular disease or COPD.

The methods described herein may also be incorporated into radiomics platforms that extract and analyze multiple quantitative features from medical images, supporting research into phenotype identification and precision medicine approaches for pulmonary disorders. By combining ArtLR with other imaging biomarkers, genetic information, and clinical data, these platforms may enable more comprehensive patient characterization than any single biomarker alone.

### Variations and Alternatives

The methods described herein may be modified in various ways without departing from the scope of the invention. Regarding image acquisition, while contrast-enhanced dual-energy CT scans were used in the described implementations, the methods may be adapted for use with standard CT scans, magnetic resonance imaging (MRI), or other modalities capable of visualizing pulmonary vessels with sufficient contrast. The choice of imaging modality and protocol may depend on available equipment, consideration of radiation exposure, contrast agent contraindications, or specific research questions.

Alternative segmentation approaches may be employed to identify pulmonary arteries. While the described implementations utilize convolutional neural networks, particularly UNet architectures, other deep learning models such as transformer-based networks, latent diffusion models, or graph neural networks may offer advantages in certain contexts. Traditional image processing techniques based on thresholding, region growing, or vesselness filters may also be suitable, particularly when deep learning resources are limited or when contrast enhancement provides sufficient differentiation between vessels and surrounding tissues.

The methods for identifying standard locations on the arterial tree may be tailored to specific applications. While three primary approaches have been described—Strahler ordering, airway correspondence, and regional entry points—other approaches may be developed. For example, locations might be defined based on distance from the main pulmonary artery, by vessel diameter ranges, or by relationship to anatomical landmarks. The optimal approach may depend on the specific research question, disease being studied, or available imaging data.

The matching algorithm for establishing correspondence between airway and arterial segments may be implemented using various machine learning techniques. While the described implementation utilizes LSTM neural networks, alternatives include attention-based models, random forests, support vector machines, or other classification approaches. The feature vectors used for matching may also be modified to include additional geometric or image-based characteristics.

The calculation of the ArtLR index itself offers several variations. The arterial dimensions may be quantified as diameters, cross-sectional areas, or volumes. The normalization factor may be based on total lung capacity, functional residual capacity, or potentially other volume measurements. The mathematical relationship between arterial dimensions and lung volume may be adjusted; while division by the cube root of lung volume was primarily described, direct division by volume or other scaling relationships might be appropriate for certain applications.

Statistical methods for correlating ArtLR with clinical parameters may vary according to the specific hypotheses being tested. While linear regression with adjustment for demographic factors was described, more complex models incorporating additional covariates, non-linear relationships, or machine learning approaches might reveal additional insights about the relationship between ArtLR and disease manifestations.

Implementation environments for the described methods may range from specialized research workstations to clinical picture archiving and communication systems (PACS) to cloud-based computing platforms. The methods may be integrated with existing pulmonary analysis software or implemented as standalone applications. User interfaces may vary from fully automated processing pipelines to interactive systems allowing expert oversight and adjustment at various processing stages.

As used herein, the term "processor" refers to any one or more devices, circuits, or combinations thereof capable of executing instructions or performing computational operations, and includes without limitation general-purpose central processing units (CPUs), graphics processing units (GPUs), tensor processing units (TPUs), neural processing units (NPUs) and other AI accelerators, digital signal processors (DSPs), field-programmable gate arrays (FPGAs), application-specific integrated circuits (ASICs), system-on-chip (SoC) devices, microcontrollers, and virtualized or cloud-based computing resources. A "processor" may comprise a single device or a plurality of devices operating in cooperation, whether co-located or distributed across multiple physical or virtual machines, and whether connected locally, over a network, or through a cloud computing environment. Recitation of "a processor" shall be understood to encompass one or more such processors acting individually or in combination to perform the recited operations. The present disclosure describes methods and systems for measuring the relative size of the pulmonary arterial tree compared to the lungs.

These variations and alternatives illustrate the adaptability of the present disclosure and the standardized measurement of arterial dimensions relative to lung size to diverse technical environments, clinical applications, and research contexts.

What is claimed is:

1. A method for assessing pulmonary vascular characteristics, comprising:
obtaining medical imaging data of a subject's lungs;
segmenting pulmonary arterial structures from the medical imaging data;
calculating a pulmonary-arterial-to-lung ratio (ArtLR) by determining a relationship between arterial dimensions at predetermined anatomical locations and lung volume; and
using the calculated ArtLR as a biomarker for assessing pulmonary structure and function.

2. The method of claim 1, further comprising segmenting a pulmonary venous tree and an airway tree from the medical imaging data.

3. The method of claim 1, wherein the predetermined anatomical locations comprise a subset of standard airway segment labels comprising at least an RB1 label and an RB4 label and wherein arterial dimensions comprise at least one of branch diameter or branch volume, measured at the predetermined anatomical locations.

4. The method of claim 1, wherein aggregated arterial measurements are normalized by a measure of lung volume to yield the arterial-to-lung ratio (ArtLR) and wherein the lung volume is determined from total lung capacity (TLC) or functional residual capacity (FRC) obtained from the medical imaging data.

5. The method of claim 1, further comprising automatically matching arterial segments to the predetermined airway anatomical locations based on metrics of geometric similarity, including proximity and orientation, wherein the automatic matching is performed using a trained machine learning model.

6. The method of claim 5, wherein the automatic matching is performed using a long short-term memory (LSTM) neural network trained to evaluate features including proximity, orientation, generational position, branch length ratio, branch diameter ratio, and airway segment identity.

7. The method of claim 1, wherein the predetermined anatomical locations are established on a pulmonary arterial tree by:
generating a graph representation of the pulmonary arterial tree having diameter information associated with branches thereof;
assigning Strahler orders to the branches based on how branches join to form upstream branches;
iteratively refining the Strahler orders using the diameter information until the ordering converges to a solution; and
selecting branches within a predetermined order range as the predetermined anatomical locations.

8. The method of claim 1, further comprising correlating the arterial-to-lung ratio (ArtLR) with at least one clinical parameter, imaging parameter, or COPD-related metric to assess pulmonary structure, pulmonary function, or susceptibility to disease.

9. A method for determining a physiological index associated with pulmonary arterial structure, the method comprising:
segmenting a pulmonary arterial tree and a pulmonary venous tree from medical imaging data using an automated process;
extracting centerlines from the segmented pulmonary arterial tree and generating a graph representation comprising nodes and edges, wherein each edge corresponds to a vascular branch and is associated with one or more geometric features including diameter and orientation;
identifying a plurality of standard airway locations from an airway tree of a subject and assigning anatomical labels to the identified airway locations;
determining a plurality of arterial segments corresponding to a subset of the standard airway locations by comparing one or more anatomical or geometric features between the arterial tree and the airway tree;
computing a physiological index based on the diameters and/or volumes of the determined arterial segments and normalizing the index based on a measurement of lung volume.

10. The method of claim 9, wherein the graph representation of the arterial tree is generated from the centerlines by associating nodes with bifurcation points and edges with vessel segments.

11. The method of claim 9, wherein determining the plurality of arterial segments comprises using a trained neural network to match each airway location to at least one candidate arterial branch based on scores based on proximity, orientation, branch generation, relative branch lengths, an airway branch identifier and/or other features.

12. The method of claim 9, wherein the subset of standard airway locations comprises nineteen locations selected based on reliability of arterial-airway correspondence.

13. The method of claim 12 wherein one of the nineteen locations is determined by averaging 5 most distal pairs of the airway locations.

14. The method of claim 9, wherein the lung volume is measured using total lung capacity (TLC) or functional residual capacity (FRC), and the normalization is performed by dividing the arterial branch metric by a lung volume metric based on the lung volume.

15. A system for calculating a pulmonary arterial-to-lung ratio (ArtLR) biomarker, comprising:
a medical imaging device configured to obtain computed tomography (CT) scans of a subject's lungs; a processor; and
a memory storing instructions that, when executed by the processor, cause the system to:
segment pulmonary arterial structures from the CT scans using a trained neural network;
extract centerlines from the segmented pulmonary arterial structures and generate a graph representation;
identify standard locations on the pulmonary arterial tree;
measure arterial dimensions at the identified standard locations; and
calculate the ArtLR biomarker by normalizing the measured arterial dimensions by a lung volume measurement.

16. The system of claim 15, wherein the trained neural network comprises a convolutional neural network architecture trained on a dataset of manually segmented pulmonary vasculature to distinguish between pulmonary arteries and pulmonary veins.

17. The system of claim 15, wherein the instructions further cause the system to identify standard locations by at least one of (a) matching arterial segments to corresponding airway segments using a machine learning algorithm that evaluates proximity, orientation, and branch characteristics or (2) based on entry points of arterial branches into sub-lobar regions of the lungs.

18. The system of claim 15, wherein the instructions further cause the system to correlate the calculated ArtLR biomarker with clinical parameters including at least one of percent emphysema, bronchovascular texture prominence, and pulmonary function measurements to assess disease risk or progression.

19. The system of claim 15, wherein the trained neural network comprises at least one of a convolutional neural network, a U-Net, a diffusion model, a transformer-based network, a Segment Anything Model (SAM), or a generative adversarial network (GAN).
